(19) United States
(12) Patent Application Publication
(10) Pub. No.: US 2025/0228772 A1
(43) Pub. Date: Jul. 17, 2025

(54) DRUG DELIVERY SYSTEM COMPRISING A REFLUX INHIBITOR FOR THE APPLICATION TO ESOPHAGEAL MUCOUS MEMBRANES

(71) Applicant: EsoCap AG, Basel (CH)

(72) Inventors: Werner Weitschies, Neuenkirchen (DE); Christoph Rosenbaum, Backnang (DE)

(21) Appl. No.: 18/872,770

(22) PCT Filed: Jun. 2, 2023
(86) PCT No.: PCT/EP2023/064785

§ 371 Date: Dec. 6, 2024

(30) Foreign Application Priority Data
Jun. 7, 2022 (EP) 22177670.1

(51) Int. Cl. A61K 9/00; A61K 31/197; A61K 47/10; A61K 47/32

(52) U.S. Cl. CPC A61K 9/006; A61K 31/197; A61K 47/10; A61K 47/32

(57) ABSTRACT

The present invention relates to a drug delivery system for the application to an esophageal mucous membrane, comprising at least one sheet like, in particular film shaped, foil shaped or wafer shaped preparation comprising a reflux inhibitor, a release mechanism, and a trigger mechanism, wherein the trigger mechanism is adapted to trigger, at a predetermined site of action the release of the sheet like preparation by the release mechanism.

### FIELD OF THE INVENTION

The present invention relates to a drug delivery system comprising a reflux inhibitor, in particular for the application to esophageal mucous membranes and for treating esophageal diseases in particular gastroesophageal reflux disease.

### BACKGROUND ART

Gastroesophageal reflux disease (GERD) is a chronic disease with phases of recurrence and remission overtime (Antunes et al., “Gastroesophageal Reflux Disease”, StatPearls Publishing, 2021). The overall prevalence of gastroesophageal reflux disease is 18-28% in the United States population, and from 9.8%-18% in Europe. Gastroesophageal reflux disease is characterized by refluxed gastric content (which can include bile and other duodeno-gastric refluxate constituents) into the esophagus. While the stomach itself is protected from gastric acid, the gastric acid is very harmful to tissues other than the stomach such as the esophagus. Typically, patients suffering from gastroesophageal reflux disease have symptoms such as heartburn, regurgitation and, rarely dysphagia. Common atypical manifestations are chronic cough or asthma, and non-cardiac chest pain. Patients can suffer from esophageal lesions or erosions which may lead to further diseases ranging from erosive esophagitis (EE) to Barrett's disease (BE).

Currently, gastroesophageal reflux disease is treated by controlling the gastric acid with proton pump inhibitors (PPIs). PPIs are able to achieve a healing rate of esophageal mucosa up to 80-90% within 8-12 weeks of treatment and lead to resolution of typical symptoms of the disease, particularly heartburn, reflux-induced chest pain, and dysphagia. However, PPIs are 20-30% less effective in non-erosive reflux disease (NERD). Moreover, there is no response on proton pump inhibitors in up to 40% of patients with persistent reflux symptoms.

A predominant mechanism underlying gastroesophageal reflux is transient lower esophageal sphincter relaxations (TLESRs) which occurs in gastroesophageal reflux disease patients. Transient lower esophageal sphincter relaxations are relaxations of the lower esophageal sphincter (LES) in absence of a swallow allowing gastric content to pass to the esophagus. The lower esophageal sphincter is a smooth muscle within the wall of the esophagus that forms a valve at the end of the esophagus where it joins the stomach and represents a therapeutic target for the local treatment of gastroesophageal reflux disease. For example, U.S. Pat. Nos. 9,925,375 and 10,661,081 describe a non-invasive device for the treatment of gastroesophageal reflux disease and the digestive system. The device is positioned on the skin of the patient's abdomen and electrically stimulates the abdomen muscles. This treatment results in dynamic motions of the digestive system, which causes the treatment of various digestive symptoms or diseases.

Drug delivery to gastrointestinal and, in particular to esophageal, membranes is usually carried out via endoscopy guided sub-membranous application. Topical application of active ingredients involve drug coated esophageal stents or oral viscous drugs. Drugs which are currently under investigation involve oro-dispersible or oro-disintegrating tablets, aerosols, or gel-like drugs with higher viscosity to increase contact time.

However, topical application of active ingredients to gastrointestinal and, in particular, esophageal membranes have some challenges. For instance, it is very difficult to locally apply high doses of a drug over a period sufficient to achieve therapeutically effective local concentrations. Possible causes of too low concentrations at the site to be treated include degeneration or deactivation of the drug by digestive secretions and enzymes, dilution effects by intestinal fluids, poor absorption, prodrugs requiring activation not available at site to be treated, and a residence time at the site of action that is too short for allowing onset of drug action effectively. Short residence times and/or too low local concentrations at the site of action are in particular a problem when using liquid or gel-like drug delivery systems. Therefore, high doses must be administered to achieve sufficient concentrations at the site to be treated. Higher administered doses of an active ingredient are usually associated with increased side effects by intestinal absorption and higher bioavailability; hence the dose of active ingredients should be kept as low as possible.

There is still a need for an appropriate drug delivery system, particularly to the esophagus, that can deliver active pharmaceutical ingredients for effective treatment while allowing administration of the lowest possible doses to reduce side effects.

### OBJECT OF THE INVENTION

It is an object of the invention to provide a drug delivery system that enables oral/topical administration of a reflux inhibitor, e.g., a GABA receptor type B agonist, for treating a gastroesophageal reflux disease with increased local efficacy.

It is a further object of the invention to provide a delivery system that allows the application of a reflux inhibitor e.g., GABA receptor type B agonist, at a low dose, thereby minimizing potential side effects.

The objects of the invention are achieved by the subject-matters of the independent claims. Preferred embodiments are subject of the dependent claims.

### SUBJECT MATTER CLAIMED

In a first aspect, the present invention relates to a drug delivery system for the application to an esophageal mucous membrane, comprising at least one sheet like, in particular film shaped, foil shaped or wafer shaped preparation comprising an active pharmaceutical ingredient; a release mechanism; and a trigger mechanism, wherein the trigger mechanism is adapted to trigger, at a predetermined site of action, the release of the preparation by the release mechanism, and wherein the release mechanism is adapted to release said preparation while moving along the esophageal mucous membrane, wherein the drug delivery system further comprises a shell, wherein the shell contains the preparation, and wherein the shell comprises an aperture as part of the release mechanism configured to allow said preparation to leave the shell, and wherein the trigger mechanism is a holding device that is a part of or is attached to the preparation, such that the preparation is unrolled or unfolded while the dosage form moves down the esophageal mucous membrane and leaves the shell through the aperture, characterized in that the active pharmaceutical ingredient comprises a reflux inhibitor, preferably a reflux inhibitor which reduces or blocks transient lower esophageal reflux relaxations, e.g., a gamma-Aminobutyric acid (GABA) receptor type B agonist.

In one embodiment, the active pharmaceutical ingredient (API) within the drug delivery system comprises a gamma-Aminobutyric acid (GABA) receptor type B agonist.

In one embodiment, the active pharmaceutical ingredient is a GABA receptor type B agonist selected from the group consisting of gamma-Aminobutyric acid (GABA) or a salt thereof, β-Phenyl-γ-aminobutyric acid (Phenibut) or a salt thereof, 4-Fluorophenibut or a salt thereof, Isovaline or a salt thereof, 3-Aminopropylphosphinic acid or a salt thereof, 3-Aminopropylmethylphosphinic acid (SKF-97,541) or a salt thereof, [3-Amino-2-hydroxypropyl]-methylphosphinic acid (CGP 44532) or a salt thereof, 4-Amino-3-(4-chlorphenyl) butyric acid (Baclofen) or a salt thereof, [(2R)-3-Amino-2-fluoropropyl] phosphinic acid (Lesogaberan) or a salt thereof, [2,6-di-tert-butyl-4-(3-hydroxy-2,2-dimethyl-propyl)-phenol] (CGP7930) or a salt thereof, (S)-3-amino-2-hydroxypropylphosphinic acid (C₃H₁₀O₃NP; AZD9343) or a salt thereof, and Arbaclofen placarbil or a salt thereof.

In one embodiment, the GABA receptor type B agonist comprises 4-Amino-3-(4-chlorphenyl) butyric acid (Baclofen) or a salt thereof.

In one embodiment, the GABA receptor type B agonist comprises the R-enantiomer of 4-Amino-3-(4-chlorphenyl) butyric acid (Baclofen) or a salt thereof.

In one embodiment, the active pharmaceutical ingredient (API) within the drug delivery system comprises a hormone such as a peptide hormone. Examples for peptide hormones are gastrin, motilin, or pancreatic polypeptide.

In one embodiment, the active pharmaceutical ingredient (API) within the drug delivery system comprises a neurotransmitter such as histamine, acetylcholine, muscarine, substance P, or bombesin.

In one embodiment, the active pharmaceutical ingredient (API) within the drug delivery system comprises other drugs such as bethanechol, carbachol, pilocarpine, metoclopramide, domperidone, cisapride, a 5-HT3-antagonist, cyproheptadine, pizotifen, or an alpha-adrenergic substance. Examples of 5-HT3-antagonists are dolasetron, granisetron, ondasetron, palonosetron, tropisetron, alosetron or ramosetron.

In one embodiment, the active pharmaceutical ingredient (API) within the drug delivery system comprises food components, e.g., a food protein.

In one embodiment, the sheet like, in particular film shaped, foil shaped or wafer shaped preparation comprising the active pharmaceutical ingredient comprises polyvinyl alcohol (PVA).

In a second aspect, the drug delivery system of the present invention is for use in therapy.

In a third aspect, the drug delivery system is for use in the treatment or prevention an esophageal disease; or for use in the treatment or prevention of an esophageal disease, which is caused by or related to gastroesophageal reflux, such as gastroesophageal reflux disease, non-erosive reflux disease, esophagitis, particularly erosive esophagitis and/or cancer, e.g., esophageal cancer such as adenocarcinoma, squamous cell carcinoma or Barrett's esophagus; or for use in the treatment or prevention of gastroesophageal reflux disease.

### DETAILED DESCRIPTION

Unless otherwise defined, all technical and scientific terms used herein have the same meaning as commonly understood by one of ordinary skill in the art to which this disclosure belongs.

Terms which are indicated in singular herein relate to the term in plural as well unless it is explicitly stated otherwise herein and vice versa.

Indefinite article “a” or “an” as used herein also relates to the definite article “the” unless indicated otherwise.

The term “comprising” or “comprises” as used herein means “including, but not limited to”. The term is intended to be open-ended, to specify the presence of any stated features, elements, integers, steps, or components, but not to preclude the presence of addition of one or more other features, elements, integers, steps, components, or groups thereof. The term “comprising” or “comprises” thus includes the more restrictive terms “consisting of” and “consisting essentially of”. In one embodiment, the term “comprising” or comprises” as used throughout the application and in particular within the claims may be replaced by the term “consisting of”.

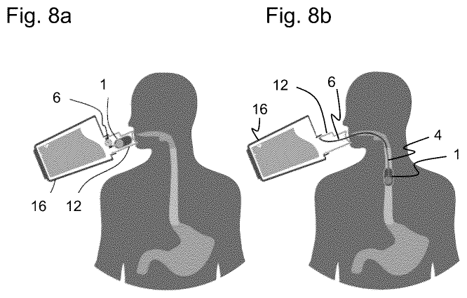

A drug delivery system comprising a pharmaceutical preparation, however with different active pharmaceutical ingredients and its application is described in PCT/EP2015/002601, which is incorporated by reference herein in full, in particular regarding the embodiment according to FIGS. 8a, 8b, 8c of PCT/EP2015/002601. Stated differently, the size, shape and composition of the shell, the aperture, the release and trigger mechanism and the holding device are at least to a significant extent already described in said reference.

The drug delivery system as described in the PCT/EP2015/002601 is designed such that it comprises at least one sheet like, in particular film shaped, foil shaped or wafer shaped preparation comprising an active pharmaceutical ingredient, a release mechanism, and a trigger mechanism, wherein the trigger mechanism is adapted to trigger, at a predetermined site of action, in particular of the gastrointestinal tract the release of the sheet like preparation by the release mechanism. From the embodiment according to FIGS. 8a, 8b, 8c of PCT/EP2015/002601, the dosage form is known to have an elongated, strip-shaped preparation, which comprises the active pharmaceutical ingredient. The preparation is capable to be arranged in a compact condition and in an expanded condition. The dosage form has a capsule device, e.g., a shell, comprising a hollow space for accommodating the compacted preparation, the capsule device has an aperture, and a first end of the preparation extends, in the compact condition, through the aperture such that the preparation can be pulled out of the hollow space into the surrounding area of the capsule thereby transferring the preparation from the compact condition to the expanded condition.

The drug delivery system according to the present invention is orally administered and improves the local availability of the reflux inhibitor. This contrasts with conventional orally administration systems, such as tablets or capsules, which are delivered via gastro-intestinal absorption into the blood circulation to the site/location to be treated only.

The local availability is improved, because the reflux inhibitor is provided in a sheet-like, in particular film-shaped, foil-shaped, wafer-shaped, or strip shaped preparation. This advantageously allows releasing the sheet-like preparation (and the reflux inhibitor being present therein) directly onto the site/location to be treated (treatment site), e.g., an esophageal mucous membrane. Thereby, preferably a large area of the sheet like preparation is exposed to the mucous membrane, i.e., to the esophageal mucous membranes. Upon exposure to the mucous membrane the sheet-like preparation releases the reflux inhibitor. Further, the preferably direct contact between the mucous membrane and the preparation results in an effective action of the reflux inhibitor at the treatment site. Due to the direct delivery of the reflux inhibitor to the treatment site, less reflux inhibitor is required resulting in reduced systemic bioavailability and reduced concentrations at neighboring, e.g., healthy areas as compared to the use of conventional preparations, such as suspensions or solutions. Further, the direct delivery to the treatment site further allows to lower the reflux inhibitor dose contained in the preparation, thereby advantageously further reducing side effects.

The drug delivery system according to the invention further advantageously allows a relatively simple and discrete handling as well as a simple, particularly space-saving storage. The reflux inhibitors, which are comprised in the drug delivery system according to the invention, have an improved stability, e.g., at high heat and humidity, when compared to solutions and gels. Usually there is no free water left in the drug delivery system according to the invention, which further improves the stability and reduces the risk of the composition becoming e.g., moldy, or otherwise unusable. Additional additives, such as preserving agents or other stabilizers, can be avoided, which is advantageous because it is known that such additives can cause allergies or further side effects.

Also, the destruction of the active pharmaceutical ingredient before it reaches the predetermined site of action, e.g., by gastric acid and/or digestive enzymes, is advantageously minimized by a drug delivery system according to the invention.

### Release Mechanism

A release mechanism relates to a mechanism which expands and releases the sheet-like preparation from a capsule device, e.g., a shell. The shell contains the sheet-like preparation in a compact form. The release mechanism releases the preparation from the shell after a trigger mechanism has/initiated the release. The release of the sheet-like preparation by the release mechanism preferably takes place by pulling the preparation at least partially out of the shell. Therefore, the sheet like preparation is adapted, such that the sheet like preparation is expandable to a predetermined extent by the release mechanism. For example, the shell contains the preparation in a folded form and the release mechanism expands the preparation from its compact, e.g., from a folded form, into its expanded, e.g., unfolded form. The release mechanism therefore causes an unfolding of the preparation. In the compact form, the preparation has a smaller spatial extent, e.g., the preparation is lumped together, coiled, or winded or brought into a smaller spatial format in another way. This also allows to provide a small dosage form, i.e., a small shell, which makes the especially oral intake of the drug delivery system more convenient for a patient. In its expanded form, the surface area of the sheet like preparation is increased by the expansion, e.g., by the unfolding of the sheet like preparation, in particular the surface area of the preparation containing the active pharmaceutical ingredient is increased. Preferably, the surface area of the preparation, in particular the surface area, which contains the active ingredient, and which contacts the esophageal mucous membrane, is in the order of the surface area of esophageal mucous membrane. The release of the preparation occurs while the shell moves down the esophageal mucous membrane. For example, during a patient swallows the dosage form, the preparation is released from the shell through an aperture. The shell therefore comprises an aperture as part of the release mechanism, configured to allow the preparation to leave the shell.

### Aperture

In this respect the aperture forms an opening in the shell, i.e., in the capsule device. In a preferred embodiment of the drug delivery system the aperture is formed as a slit. A slit is arranged such that the sheet-like preparation is released from the shell through the aperture. Such a slit may be embodied in different arrangements and configurations. Such an aperture is described in, for example, in EP21175427.0, EP21175436.1, PCT/EP2015/002601 and PCT/EP2020/056934, which are incorporated by reference herein in full, regarding the capsule device and the aperture.

### Trigger Mechanism/Holding Device

The drug delivery system comprises a trigger mechanism, wherein the trigger mechanism is adapted to trigger, at a predetermined site of action, the release of the sheet like preparation by the release mechanism, wherein the trigger mechanism is a holding device that is part of or is attached to the preparation.

Preferably, the preparation comprises the holding device, further preferably, the preparation comprises the holding device at one end of the preparation, which, in particular protrudes out of the shell through the aperture. Upon fixation of the holding device, the preparation can be withdrawn from the capsule device by a pulling movement and/or force. Fixation of the holding device is obtained by preferably connecting the holding device to a retainer. Such a retainer can be a string member, as for example, a cord, string, or tether. In a preferred embodiment, the holding device is connected to one end of the preparation and to one end of the cord, whereas the other end of the cord is secured to an applicator, e.g., to a holder of the applicator.

Preferably, the holding device is attached to the sheet like preparation. Thereby the retainer, i.e., the string member or a part of the string member form the holding device. For example, the one end of the cord which is connected to the preparation forms the holding device.

Alternatively, the holding device is adapted to be fixed in the oral cavity or the holding device is adapted to be held in hand during administration of the drug delivery system, such that the preparation is unrolled and or unfolded while the dosage form moves down the esophageal mucous membrane and leaves the shell through the aperture.

In a preferred embodiment, a part of the string member is connected to an end portion of the preparation, which protrudes from the aperture of the capsule device. Thereby the holding device is formed by the protruding end portion of the preparation and the string member being connected to it and the further part of the string member acts as a retainer, to retain the holding device from moving while swallowing the preparation, thereby creating a pulling force which acts onto the preparation, and which pulls the preparation out of the capsule device while the capsule device moves down the esophagus.

It is to be understood that the terms “site of action” and “application site” as used herein are used interchangeably. In this regard, it is also to be understood that “site of action” and “site of application” refer to the predetermined location of release of the preparation. Moreover, an active pharmaceutical ingredient, which is released at the “site of action” respectively “application site” may exert its actual biochemical effect also at another location of the body or at another site of a biochemical cycle, e.g., at or after metabolization by the liver or reaching of an active pharmaceutical ingredient at its target molecule. “Site of action” and “application site” as used herein do not necessarily refer to the location of the biochemical, medical effect of the active pharmaceutical ingredient.

### Capsule Device/Shell

The drug delivery system according to the present invention further comprises a shell, wherein the shell contains the at least one sheet-like, in particular film-shaped, foil-shaped, or wafer-shaped preparation comprising the active pharmaceutical ingredient, and wherein the shell comprises the aperture as part of the release mechanism configured to allow said preparation to leave the shell, such that the preparation is unrolled or unfolded while the dosage form moves down the esophageal mucous membrane and leaves the shell through the aperture. The shell may further be prepared such that it protects the preparation against an unwanted release. The shell is a capsule device and, in particular, has the shape of a capsule.

In preferred embodiment, the shell comprises a first halve-capsule shell and a second halve capsule shell, and the capsule device is formed by sliding the first halve-capsule shell into the second halve-capsule shell to a joined position, such that the aperture is formed in the joined position by the second halve capsule shell overlapping a cross section of an opening, which is located in the first halve-capsule shell.

In a further embodiment, the two capsule-halves are telescoped into each other, whereas the opening of the first halve-capsule shell is covered by a further provided overlapping wall part, e.g., a patch or a tape, which is attached to the first and or second halve capsule shell.

In an alternative embodiment, the capsule halves are shaped like two nutshells and positioned on top of each other to form the capsule. The aperture is formed by a cutout, particularly at the edge of one of the two shells. Alternatively, cutouts can be formed on the edges of both halves, which when positioned and aligned on top of each other form the aperture.

In a preferred embodiment of the drug delivery system according to the present invention the shell is made out of a material that is selected from the group comprising hard gelatin, polymers, thermoplastics as e.g., Eudragit or the like. In this regard, in particular, materials can be beneficial that have been successfully tested, used and/or authorized already, e.g., for oral dosage forms.

Such a capsule device or shell is further described, for example, in EP21175427.0, EP21175436.1 and PCT/EP2020/056934, which are incorporated by reference herein in full, with regard to the capsule device.

### The Conditions to be Treated

The drug delivery system described herein is for use in therapy. In one embodiment, it is adapted for the treatment and prevention of esophageal diseases, exemplary esophageal diseases caused by or related to gastroesophageal reflux, such as gastroesophageal reflux disease, non-erosive reflux disease, esophagitis, particularly erosive esophagitis and/or cancer, such as esophageal cancer, e.g., adenocarcinoma, squamous cell carcinoma and in particularly Barrett's esophagus. In one embodiment the drug delivery system according to the invention is adapted for the treatment and/or prevention of gastroesophageal reflux disease. Further, the drug delivery system is adapted for the treatment and/or prevention of non-erosive reflux disease. Further, the drug delivery system is adapted for the treatment and/or prevention of erosive esophagitis. Further, the drug delivery system is adapted for the treatment and/or prevention of cancer, such as adenocarcinoma, particularly Barrett's esophagus.

Barrett's esophagus is a serious complication of gastroesophageal reflux disease. In Barrett's esophagus, normal tissue lining the esophagus is replaced by tissue that resembles the lining of the intestine. Barrett's esophagus does not have any specific symptoms, although patients with Barrett's esophagus may have symptoms related to gastroesophageal reflux disease. It does, though, increase the risk of developing esophageal adenocarcinoma, which is a serious, potentially fatal cancer of the esophagus. Diagnosis of Barrett's esophagus may be done using endoscopy, histology, and/or using biomarkers, for instance as described in US20120009597 A1.

Within the subject application, the term “treatment and/or prevention” includes any way of ameliorating a certain condition or disease to be treated or preventing the condition or disease to be treated to occur. It also includes the prevention of a worsening of the condition or disease and minimizing the severity of the condition or disease.

As used herein, esophageal cancer refers to cancer that starts in the esophagus, including but not limited to squamous cell carcinoma and adenocarcinoma.

A predominant mechanism underlying gastroesophageal reflux is transient lower esophageal sphincter relaxations which occurs in gastroesophageal reflux disease patients. “Transient lower esophageal sphincter relaxation” or “TLESR” as used herein refer to a relaxation of the lower esophageal sphincter in absence of a swallow. Thus, transient lower esophageal sphincter relaxations might result in inappropriate opening of the lower esophageal sphincter allowing gastric acid to enter the esophagus. Without being bound to any theory, transient lower esophageal sphincter relaxations might be triggered by specific receptors located on nerves within the stomach muscle layer or by receptors located in the within the gastric mucosa.

Rohof et al. (Neurogastroenterol. Motil. (2012) 24:383-391) identified gamma-Aminobutyric acid (GABA) receptors type B (also referred to as GABAB receptor) in neurons of the human myenteric plexus of the lower esophageal sphincter. Hyland et al. (Frontiers in Pharmacology 1 (2014), Article 124:1-9) report on GABAB receptor subunit 1 expression on myenteric and submucosal neurons in mouse colon and ileum. Further, a meta-analysis of nine clinical studies has found that Baclofen, a GABAB receptor agonist, decreased the number and the length of reflux episodes as well as the incidence of transient lower esophageal sphincter relaxations (Vela M F, Tutuian R, Katz P O, Castell D O. Aliment Pharmacol. Ther. 2003 January; 17 (2): 243-51, Li S, Shi S, Chen F, Lin J. Gastroenterol Res Pract. 2014; 2014( ):307805, Wise J, Conklin J L. Curr. Gastroenterol. Rep. 2004 June; 6 (3): 213-9). Moreover, Baclofen was found to control transient lower esophageal sphincter relaxations after systemic administration (Lidums, Gastroenterology, 118:2000; 7-13). Thus, GABAB receptor agonists, and particularly Baclofen, are promising candidates for the drug delivery device of the invention.

### Active Pharmaceutical Ingredient

Within the drug delivery system as claimed, reflux inhibitors, e.g., GABA receptor type B agonists; a hormone such as gastrin, motilin, histamine or pancreatic polypeptide; a neurotransmitter such as histamine, acetylcholine, muscarine, substance P, or bombesin; another drug such as bethanechol, carbachol, pilocarpine, metoclopramide, domperidone, cisapride, a 5-HT3-antagonist, cyproheptadine, pizotifen, or an alpha-adrenergic substance; or a food component, are used as the main active pharmaceutical ingredient(s).

The term “active pharmaceutical ingredient” as used herein is used interchangeably with the term “active ingredient” or API and refers to a reflux inhibitor. Further APIs that might be present in addition to the reflux inhibitor are referred to herein as “additional active pharmaceutical ingredient” or “additional active ingredient” or “additional API”.

By the term “therapeutically effective dose” or “effective amount” is meant a dose or amount that produces the desired effect for which it is administered. The exact dose or amount will depend on the purpose of the treatment and will be ascertainable by one skilled in the art using known techniques. The term “therapeutically effective amount” is an amount that is effective to ameliorate (a symptom of) a disease. A therapeutically effective amount can be a “prophylactically effective amount” as prophylaxis can be considered therapy.

In one embodiment, the active pharmaceutical ingredient is a reflux inhibitor.

A “reflux inhibitor” as used herein includes but is not limited to a compound which interferes with, reduces, inhibits and/or blocks reflux, particularly gastroesophageal reflux. In a specific embodiment the reflux inhibitor interferes with, reduces, inhibits and/or blocks transient lower esophageal reflux relaxations. In one embodiment, the reflux inhibitor increases or improves the lower esophageal pressure, thereby preferably reducing, inhibiting and/or blocking transient lower esophageal sphincter relaxations. In one embodiment, the reflux inhibitor improves the function, e.g., improves or increases the contractility, of the smooth muscle of the lower esophageal sphincter, thereby preferably reducing, inhibiting and/or blocking transient lower esophageal sphincter relaxations. In one embodiment, the reflux inhibitor increases the distal esophageal sphincter function. In a preferred embodiment, the reflux inhibitor is a GABA receptor type B agonist. In one embodiment, the reflux inhibitor is a protein, e.g., an antibody, such as an antibody capable of activating GABA receptor type B or a hormone or a neurotransmitter or a food protein. A protein might be mammal protein, preferably a human or mouse protein. A protein might be administered as active protein or inactive protein precursor, pre-protein, or pro-protein. A protein might be administered as nucleic acid encoding the protein. In one embodiment, the reflux inhibitor is a small molecule, e.g., a small molecule with a molecular mass of about 900 g/mol or less. In one embodiment, the reflux inhibitor is a hormone such as gastrin, motilin, histamine or pancreatic polypeptide; a neurotransmitter such as histamine, acetylcholine, muscarine, substance P, or bombesin; another drug such as bethanechol, carbachol, pilocarpine, metoclopramide, domperidone, cisapride, a 5-HT3-antagonist, cyproheptadine, pizotifen, or an alpha-adrenergic substance; or a food component.

In one embodiment, the active pharmaceutical ingredient is a GABA receptor type B agonist or a salt thereof.

A “GABA receptor type B agonist” as used herein refers to a compound that activates the GABA receptor type B to produce a biological response and includes but is not limited to an endogenous agonist, i.e., an agonist that is naturally produced by the body and activates the receptor, such as GABA, or an exogenous agonist, such as a drug, e.g. β-Phenyl-γ-aminobutyric acid (Phenibut) or a salt thereof, 4-Fluorophenibut or a salt thereof, Isovaline or a salt thereof, 3-Aminopropylphosphinic acid or a salt thereof, 3-Aminopropylmethylphosphinic acid (SKF-97,541) or a salt thereof, [3-Amino-2-hydroxypropyl]-methylphosphinic acid (CGP 44532) or a salt thereof, 4-Amino-3-(4-chlorphenyl) butyric acid (Baclofen) or a salt thereof, [(2R)-3-Amino-2-fluoropropyl] phosphinic acid (Lesogaberan) or a salt thereof, [2,6-di-tert-butyl-4-(3-hydroxy-2,2-dimethyl-propyl)-phenol] (CGP7930) or a salt thereof, (S)-3-amino-2-hydroxypropylphosphinic acid (C₃H₁₀O₃NP; AZD9343) or a salt thereof, and Arbaclofen placarbil or a salt thereof.

In one embodiment, the GABA receptor type B agonist, preferably an exogenous agonist, is an agonist which mimics the response of the endogenous agonist at least partially. In one embodiment, the GABA receptor type B agonist, preferably an exogenous agonist activates the receptor with a response comparable to the endogenous agonist, e.g., GABA. In a specific embodiment, the agonist is a selective agonist, i.e., it is specific for the GABA receptor type B. In another embodiment, the GABA receptor type B agonist is a co-agonist, e.g., an agonist which works together with a further (co-) agonist to produce the response.

A “biological response” as referred to herein relates to any biological response of the GABA receptor type B. In one embodiment, the biological response is those evoked by GABA. In another embodiment, the biological response is a modulation, e.g., inhibition or activation, of neurotransmitter release. In another embodiment, the biological response is modulation of neurotransmitter release by depressing Ca²⁺ influx, preferably presynaptically. In another embodiment, the biological response is modulation of neurotransmitter release by coupling inwardly rectifying K⁺ channels and mediate slow inhibitory postsynaptic potentials, preferably postsynaptically.

In one embodiment, the GABA receptor type B agonist is selected from the group consisting of gamma-Aminobutyric acid (GABA) or a salt thereof, β-Phenyl-γ-aminobutyric acid (Phenibut) or a salt thereof, 4-Fluorophenibut or a salt thereof, Isovaline or a salt thereof, 3-Aminopropylphosphinic acid or a salt thereof, 3-Aminopropylmethylphosphinic acid (SKF-97,541) or a salt thereof, [3-Amino-2-hydroxypropyl]-methylphosphinic acid (CGP 44532) or a salt thereof, 4-Amino-3-(4-chlorphenyl) butyric acid (Baclofen) or a salt thereof, [(2R)-3-Amino-2-fluoropropyl] phosphinic acid (Lesogaberan) or a salt thereof, [2,6-di-tert-butyl-4-(3-hydroxy-2,2-dimethyl-propyl)-phenol] (CGP7930) or a salt thereof, (S)-3-amino-2-hydroxypropylphosphinic acid (C₃H₁₀O₃NP; AZD9343) or a salt thereof, and Arbaclofen placarbil or a salt thereof.

In a preferred embodiment, the GABA receptor type B agonist comprises 4-Amino-3-(4-chlorphenyl) butyric acid (Baclofen), preferably (RS)-4-Amino-3-(4-chlorphenyl) butyric acid.

In a further preferred embodiment, the GABA receptor type B agonist comprises 4-Amino-3-(4-chlorphenyl) butyric acid (Baclofen) and has the following formula:

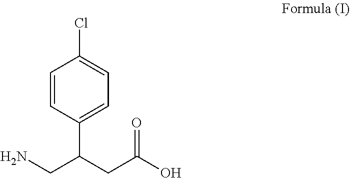 [SMILES: NCC(CC(=O)O)c1ccc(Cl)cc1]

or a salt thereof.

In one embodiment, the GABA receptor type B agonist comprises the R-enantiomer of 4-Amino-3-(4-chlorphenyl) butyric acid (Baclofen) or a salt thereof.

In one embodiment, the active pharmaceutical ingredient (API) comprises or is a hormone, such as histamine and/or a peptide hormone. Examples for peptide hormones include but are not limited to gastrin (e.g., UniProt P01350), motilin (e.g., UniProt P12872), and/or pancreatic polypeptide (e.g., UniProt P01298).

In one embodiment, the active pharmaceutical ingredient (API) comprises or is a neurotransmitter. Examples of neurotransmitters include but are not limited to histamine, acetylcholine, muscarine, substance P (e.g., UniProt P20366), and/or bombesin (e.g., UniProt P84214). In one embodiment, the neurotransmitter is preferably bombesin.

In one embodiment, the active pharmaceutical ingredient (API) comprises or is another drug such as bethanechol, carbachol, pilocarpine, metoclopramide, domperidone, cisapride, a 5-HT3-antagonist, cyproheptadine, pizotifen, and/or an alpha-adrenergic substance. Examples of 5-HT3-antagonists are dolasetron, granisetron, ondasetron, palonosetron, tropisetron, alosetron or ramosetron.

In one embodiment, the active pharmaceutical ingredient (API) comprises or is a food component, e.g., a food protein.

In one embodiment, the active pharmaceutical ingredient is a protein and might be administered as active protein.

In one embodiment, the active pharmaceutical ingredient is a protein and might be administered as inactive protein, e.g., as precursor protein, pre-protein, or pro-protein. In such cases, the inactive protein will be converted into its active form, e.g. after administration.

In one embodiment, the active pharmaceutical ingredient is a protein and might be administered as nucleic acid encoding the protein.

The administration frequency of the drug delivery device and the treatment period or the time point of administration is not limited and may be dependent of the specific disease to be treated and/or the amount and/or nature of the active pharmaceutical ingredient per drug delivery device. For example, the drug delivery device can be administered once per day or twice day. If the drug delivery device is administered once a day, it is preferably administered in the evening to increase the patient's compliance. The drug delivery system of the invention is preferably administered before bedtime, i.e., after dinner and after oral hygiene. The treatment period may be between 7 days to 40 days, preferably 14 days to 30 days, more preferably from 20 days to 28 days. The treatment may comprise a single treatment cycle of the treatment period or multiple cycles, e.g., 2, 3, 4, 5, 6, 7, 8, 9, 10 or more treatment periods.

The present invention provides a drug delivery system comprising an, i.e., one active pharmaceutical ingredient or a combination of active pharmaceutical ingredients or an active pharmaceutical ingredient as described herein and an additional active pharmaceutical ingredient.

### Additional Active Pharmaceutical Ingredients

The API within the present dosage form may be administered together with an additional API. Such additional APIs may be selected from the skilled person based on his or her general knowledge depending upon the condition to be treated and/or prevented.

### Preparation

In a preferred embodiment of the drug delivery system according to the present invention the sheet like preparation is a wafer or is formed as a wafer. The term “wafer” as used herein, refers to a sheet, which comprises several layers used to enclose the active pharmaceutical ingredient.

Such a wafer can fit to the irregular surface contour of a predetermined site of action, in particular of the esophageal mucous membrane, in particular after absorption of moisture contained in the esophageal mucous membrane by the wafer. Additionally, a sheet like preparation of a dosage form according to the invention may be gellable or swellable.

In a preferred embodiment of the drug delivery system according to the present invention the thickness of the sheet like preparation is 0.01 mm to 2 mm, preferably 0.03 mm to 1 mm, preferably 0.05 mm to 0.1 mm. This is beneficial to provide a sheet like preparation with a relatively small thickness.

In a preferred embodiment of the drug delivery system according to the present invention the sheet like preparation has an area between 0.5 and 25 cm², preferably between 1 to 10 cm².

The sheet like preparation may have different shapes. In particular, a sheet like preparation can have a round, triangular, quadrangular or polygonal shape. In an embodiment, the aperture is adapted to fit the respective shape of the preparation.

In a preferred embodiment of the drug delivery system according to the present invention the sheet like, in particular film shaped, foil shaped, or wafer shaped, preparation, that comprises the active pharmaceutical ingredient, contains an active pharmaceutical ingredient with a drug content of 0.0001 to 50% by weight, preferably 0.001 to 25% by weight, and most preferred 0.01 to 10% by weight.

The sheet like preparation comprising the active pharmaceutical ingredient may have a single-layered or multi-layered structure, wherein at least one (preferably first) layer contains the active pharmaceutical ingredient.

In a preferred embodiment the sheet like preparation has a multi-layered structure of multiple layers, wherein at least a first layer contains the active pharmaceutical ingredient and wherein at least a further layer contains at least one further active pharmaceutical ingredient, which is either the same active pharmaceutical ingredient or an additional active pharmaceutical ingredient.

In a preferred embodiment the layer containing the active pharmaceutical ingredient and/or the further layer containing the additional active pharmaceutical ingredient comprises a polymer, preferably a film forming polymer.

The polymer within the layer may serve merely as a carrier for the active pharmaceutical ingredient and/or the additional API, or it may serve as a reservoir for same. Such a layer can release the active pharmaceutical ingredient and/or the additional active pharmaceutical ingredient under the effect of a fluid. The active pharmaceutical ingredient and/or the additional API may be released immediately or in a controlled release manner.

In a preferred embodiment of the drug delivery system according to the present invention the sheet like preparation comprises at least a first layer containing the active pharmaceutical ingredient and/or a further layer containing the active pharmaceutical ingredient and/or additional API, wherein the at least one first layer and/or the further is an adhesive layer.

In a preferred embodiment of the drug delivery system according to the present invention the at least one first layer containing the active ingredient and/or the further layer containing the active ingredient comprises a polymer, preferably a film forming polymer, wherein the polymer is a film forming polymer that is water dispersible and/or decomposable and/or water disintegrable.

A polymer for a first layer containing an active substance and/or for a further layer containing an active substance may, in particular, be selected from a group comprising polyvinyl alcohols, Polyvinylpyrrolidone, polyvinyl acetate, polyethylene glycol, polyethylene oxide polymers, polyurethanes, polyacrylic acids, polyacrylates, polymethacrylates, poly (methyl vinyl ether-maleic acid anhydrides), starch, starch derivates, natural gums, alginates, pectins and gelatin, Pullulan, gel forming proteins, Chitosan, Agar-Agar, agarose, carrageenan, xanthan, tragacanth, dextran, and cellulose ethers such as ethyl cellulose, hydroxyethyl cellulose, propyl cellulose, carboxymethyl cellulose, sodium-carboxy methylcellulose, hydroxypropyl cellulose, hydroxypropyl methylcellulose, hydroxypropyl ethyl cellulose, cellulose acetate, povidone and copovidone. In a preferred embodiment, the polymer is polyvinyl alcohol, more preferably polyvinyl alcohol 18-88.

The polymers may be used individually or in a combination with each other to manufacture a sheet like preparation for the dosage form according to the invention with the desired properties as adhesion, release or disintegration properties. A sheet like preparation according to the invention may consist of a single polymer layer.

Also, a sheet like preparation for a dosage form according to the invention may have a structure with two or multiple layers, when at least one of the layers contains an active pharmaceutical ingredient. It is also possible that multiple layers contain either the active pharmaceutical ingredient or an additional API.

In a preferred embodiment of the drug delivery system according to the present invention, the sheet like preparation comprising the reflux inhibitor, comprises or consists of a single-layered structure, wherein a (preferably first) layer contains the reflux inhibitor, preferably the GABA receptor type B agonist or a salt thereof, more preferably Baclofen. The layer comprises a polymer, preferably a film forming polymer, wherein the polymer is a film forming polymer that is water dispersible and/or decomposable and/or water disintegrable. The polymer is a polymer as described herein, preferably polyvinyl alcohol, preferably polyvinyl alcohol 18-88. The layer further comprises an additive as described herein, such as a plasticizer, preferably glycerol.

In another preferred embodiment of the drug delivery system according to the present invention the sheet like preparation comprising the active pharmaceutical ingredient comprises at least one first active ingredient free layer that does not contain the active pharmaceutical ingredient.

In a preferred embodiment of the drug delivery system according to the present invention the sheet like, in particular film shaped, foil shaped, or wafer shaped, preparation comprising the active pharmaceutical ingredient comprises at least a further active ingredient free layer that does not contain the active pharmaceutical ingredient.

In a preferred embodiment of the drug delivery system according to the present invention the first active ingredient free layer and/or the at least one further active ingredient free layer is a water insoluble layer which preferably comprises water insoluble substances selected from the group ethyl cellulose and/or combinations of ethyl cellulose with other water insoluble substances, hydrophobic plasticizers, especially triethyl citrate, and/or dies and/or fragrances and/or flavorings.

In particular, the use of ethyl cellulose may be beneficial due to its properties comprising a good processability, biocompatibility, and water insolubility.

In a preferred embodiment of the drug delivery system according to the present invention the first active ingredient free layer and/or the at least one further active ingredient free layer is an adhesive layer of desired thickness.

The adhesive layer may be a mucoadhesive polymer selected from the group comprising cellulose derivates, such as hydroxypropyl cellulose, starch, and starch derivates, polyvinyl alcohol, polyethylene oxide, polyethylene, polypropylene, polyacrylic acid and polyacrylate derivates, polyvinylpyrrolidone, povidone, copovidone, sodium alginate, gelatin, xanthan gum, carrageenan, pectins, dextrans, lectins, chitosan, pullulan, and mixtures thereof.

Additionally, or alternatively, the adhesive layer may comprise a solvent that is selected from the group comprising water, ethanol, methanol, acetone, organic solvents, and mixtures thereof.

Furthermore, the preparation may additionally contain additives such as colorants, fragrances, flavoring agents, preservatives, antioxidants, penetration enhancers, solubilizers, disintegration accelerators, pore formers, lubricants, and mixtures thereof. In particular, the following substances are eligible as additives: lubricants, lubricants, glidants, binders, additional active ingredients, disintegrants, antioxidants, chelating agents, coating agents, flow agents, preservatives, fillers, surfactants, plasticizers, and pigments. Furthermore, the additives may be selected from the following group: pore formers, penetration enhancers, solubilizers, emulsifiers, comprising polyethoxylated sorbitan fatty acid esters, ethoxylated fatty alcohols, and lecithin; plasticizers, comprising polyethylene glycol, glycerol and other polyhydric alcohols, higher alcohols such as dodecanol, undecanol, or octanol, sorbitol, mannitol and other sugar alcohols, dexpanthenol and triglycerides; fillers comprising highly disperse silicon dioxide, titanium dioxide, zinc oxide, chalk and starch; colorants; sweetening and flavoring agents; wetting agents; preservatives; pH regulators and antioxidants; disintegration accelerators; penetration enhancers which improve the resorption of the active pharmaceutical ingredient into the mucous membrane, such as the cellular uptake, for example, fatty acids, salts thereof, and fatty acid esters, preferably saturated fatty acids such as octanoic acid (C8), decanoic acid (C10), octadecanoic acid (C18), or unsaturated fatty acids such as oleic acid (C18), salcaprozate (SNAC) or a salt thereof, terpenes, glycolipids, medium-chained triglycerides, synthetic waxes such as isopropyl myristate, branched fatty alcohols such as Eutanol GR, urea, polypropylene glycol, dimethyl sulfoxide, azones, azone analogs, polyhydric alcohols such as propanediol, tocopherols or essential oils such as menthol. A preferred plasticizer is glycerol.

The sheet-like preparation may further comprise at least one taste-masking additive. This advantageously allows the masking of a bitter or in some other way unpleasant tasting active pharmaceutical ingredient but may also be beneficial to accelerate the onset of effect of an active pharmaceutical ingredient. Taste-masking additives are known to the person skilled in the art. Such a taste-masking additive may, in particular, comprise a sugar alcohol selected from mannitol, sorbitol, xylitol, malitol, lactitol, erythritol, threitol, and isomalt as well as sodium hydrogen carbonate.

In particular, the additives may improve the local availability of the active ingredient, such as penetration enhancers.

According to a preferred embodiment the drug delivery system, in particular the sheet like preparation, according to the invention is intended to enable a time delayed active ingredient release. The active pharmaceutical ingredient is preferably released over a period of 4 hours, preferably over a period of 6 hours and most preferably over a period of 8 hours. In order to achieve a delayed active ingredient release in case of two-layered or multi-layered preparations, at least one of the layers containing an active pharmaceutical ingredient, in particular a polymer layer, has a delayed active ingredient release.

For a delayed active ingredient release the film shaped preparations are preferably formulated as slowly soluble or slowly disintegrating film which are completely disintegrated or dissolved only after several hours. Preferably, they are completely disintegrated or completely dissolved only after 4 hours, preferably only after 6 hours, and even most preferably only after 8 hours or even only after 24 hours.

In particular, the active pharmaceutical ingredient and the optionally present additional API are released within a period of 15 minutes to 24 hours, 2 hours to 24 hours, 3 hours to 12 hours, 4 hours to 8 hours, or 5 to 6 hours.

The sheet like preparation can be prepared by a person skilled in the art by basically known methods, for example by coating of an inert support with a liquid composition which comprises the polymer(s), active pharmaceutical ingredient/additional active pharmaceutical ingredient(s) and optionally additive(s) and solvent(s), by means of e.g., a method involving a doctor blade (e.g., solvent casting), spray processors or extrusion processors. The thin film layer obtained in such a way is dried. For a multi-layered sheet like preparation one or more coatings may be applied onto the existing film layer in the same manner or may be manufactured separately and then be subsequently laminated.

During manufacture of the preparation, the temperature-sensitivity of the used active pharmaceutical ingredient needs to be taken into consideration. Thus, and in addition in view of the low dosages needed, impregnation processes may be used. In such processes an active pharmaceutical ingredient is merely applied, e.g., sprayed or dripped, onto a polymer film, which is ultimately dried.

Alternatively, the active pharmaceutical ingredient solution may be applied to the polymer film via an inkjet process.

In a preferred embodiment, the active pharmaceutical ingredient may be incorporated such that it is embedded in the polymer film, e.g., by solvent casting. Such methods are known to the skilled person and are described in the examples provided herein.

In all these processes care should be taken with regard to the solvent used, and the drying conditions. As a very elegant drying method evaporation or freeze-drying may be used.

Furthermore, depending on the stability of the active pharmaceutical ingredient incorporated also melt extrusion of polymer and active pharmaceutical ingredient are imaginable e.g., as described in Example 2 herein.

In one embodiment, the preparation is manufactured such that the active pharmaceutical ingredient is only present in certain portions within the film, which would allow a tailor-made treatment of the mucosa in designated areas only. In one specific embodiment, the preparation is manufactured such that the active pharmaceutical ingredient is only present in one or more portion(s) suitable to administer the active pharmaceutical ingredient to the lower esophageal sphincter.

Alternatively, and preferably, a first region of the sheet like preparation may be in contact with an esophageal mucosa and a second region of the sheet like preparation may be in contact with a buccal mucosa. In this way, the esophageal mucosa can be treated with the active pharmaceutical ingredient while the buccal mucosa is treated with a second active pharmaceutical ingredient, an additional API, not treated or an additive is released to the buccal mucosa. In particular, a flavoring agent and/or a local anesthetic may be released, particularly to increase or decrease the production of saliva and/or to make the application of the drug delivery system more pleasant and/or to suppress the urge to gag. Alternatively, the first region of the sheet like preparation may be in contact with an esophageal mucosa and the second region of the sheet like preparation may be in contact with the mucosa of the upper section of the stomach, such as the cardia, or the cardia and the fundus. It would therefore be possible to treat the esophagus and parts of the stomach locally.

In another preferred embodiment, the drug delivery system, in particular the capsule device, comprises a sinker device. The sinker device is configured to provide negative buoyancy to the capsule device. In experiments of the inventors underlying the finding of this preferred embodiment it was found that reducing the buoyancy, for example by increasing the mass of the capsule device, leads to an improved reliability of the mechanical process of expanding the preparation from the compacted condition to the expanded condition. In case of strip-like preparation, the unwinding of the preparation from the compacted condition, where the strip-like preparation is wound around a winding axis, to the expanded condition was significantly facilitated and more efficient. The problem underlying the preferred embodiment is the observation that the transfer of the preparation from the compacted condition to the expanded condition is sometimes incomplete. While the invention already improves the efficiency of expansion, or respectively, unwinding, by providing a spacing between the opening and the preparation, the sinker device additionally increases the efficiency of expansion. It is assumed that the capsule device, also if properly swallowed by a patient in the presence of water or aqueous solution, is not completely filled with water but air-bubbles sometimes remain inside the capsule device. The air contributes to buoyancy, and the sinker device assists to resist the buoyancy effects by assisting in the displacement of air or by using denser materials than water for utilizing gravity. Further details regarding the sinker are to be deduced from WO2020/183005, which is incorporated herein by reference.

A preferred embodiment of the drug delivery system according to the present invention is adapted for the application to a nasopharyngeal mucosa.

When the sheet like preparation releases the active pharmaceutical ingredient, optionally together with an additional API, locally and/or over a prolonged time, the therapeutic response may be improved, and in particular the local effect of the active pharmaceutical ingredient can be increased by e.g., a penetration enhancer. Such penetration enhancers are known in the art. Furthermore, in particular due to the spatially extended region of action, the necessity for a systemic administration may be reduced.

In a preferred embodiment of the drug delivery system according to the present invention the sheet like preparation has an area and/or surface area between 0.5 and 25 cm², preferably between 2 to 25 cm², preferably between 5 to 25 cm², preferably between 5 to 15 cm², preferably larger than 0.5 cm², and preferably smaller than 40 cm². Preferably the ration of the length of the sheet like preparation and the width of the sheet like preparation is between 40:1 and 400:1, or preferably 60:1 and 300:1, or preferably 80:1 and 200:1. Said width can be an average width of the sheet like preparation, measured, for example, perpendicular to the length of the sheet like preparation. Said ratio can be a ratio of the length of the sheet like preparation and a circumference, in particular an average, of the sheet like preparation, wherein said circumference can be, for example, twice the width of a sheet like preparation in the case of a strip-shaped sheet like preparation.

In certain embodiments of the drug delivery system according to the present invention the sheet like preparation is in a solid-state, in particular while it is in its compact form and/or immediately after its release. This may beneficially enhance, enable or facilitate some of the above-mentioned advantages. In particular, this may enhance the storability, when it is in a solid state prior the release. In particular, this may enhance and/or enable a targeted and/or sustained release of the active pharmaceutical ingredient, when it is in a solid state after its release. Additionally, or alternatively, in certain embodiments of the drug delivery system according to the present invention the sheet like preparation is adapted to dissolve, e.g., bio-degenerate, immediately, after a delay, in a time-controlled manner or upon a stimulus after its release. This may beneficially enhance, enable or facilitate some of the above-mentioned advantages. In particular, this can improve the user convenience, because the sheet like preparation does not need to be removed.

Additionally, or alternatively, in certain embodiments of the dosage form according to the present invention the sheet like preparation is adapted to dissolve, e.g., to bio-degenerate, preferably in a time-controlled manner, e.g., within one hour, or within one to two hours or within one to five hours, or within one to twelve hours, or within one to twenty-four hours. This improves the user convenience as the sheet like preparation does not need to be removed.

### Applicator/Retainer

In an embodiment, an applicator with a holder serves to assist swallowing the capsule device in combination with a drinking cup. The applicator in combination with the drinking cup allows the patient to take the drug delivery system as if drinking from a bottle. The applicator is therefore mounted on the drinking cup as a mouthpiece. The drug delivery system is positioned in the holder of the applicator. When drinking, the liquid of the drinking cup is rinsed through the applicator and the holder inside, which releases the preparation from the holder and transports it into the mouth of the patient, who then swallows it. The string member is a retainer and is wound around the holder. The string member is further connected to the holder and to the end of the preparation, which extends through the aperture. Thus, when the preparation leaves the holder during drinking, the holder is unwound until it is taut. This then exerts a force on the preparation, pulling the preparation out of the capsule.

Such an applicator and drinking cup is, for example, described in PCT/EP2020/056927, which is incorporated by reference herein in full, with regard to an applicator, a drinking cup and a string. Such a retainer is further described, for example, in EP21175427.0 and EP21175436.1, which are incorporated by reference herein in full, with regard to a retainer.

In a preferred embodiment, the retainer is wrapped around a support structure of the holder, whereas one end of the retainer is attached to the support structure and the other end is connected to the preparation of the capsule device. The capsule device is therefore positioned and hold inside the holder of the applicator. When the patient swallows the dosage form, the retainer begins to unwind from the support structure. The applicator and the support structure have a cylindrical shape so that the support structure fits into the applicator and, in particular, is rotatably mounted therein so that the retainer can unwind from the structure by rotating the structure.

This is particularly beneficial, if the dosage form is to be administered on a regular, in particular daily, basis as administration of the capsule device is then possible without professional help.

In a preferred embodiment of the drug delivery system according to the present invention, the release mechanism comprises the retainer, which preferably is a string, wherein the string is expandable from a compact form to an expanded form and connected to an end of the preparation which protrudes out of the capsule device.

Exemplary embodiments of the present invention will be described in greater detail below with reference to the accompanying drawings and samples, from which further features, advantages, and embodiments can be learned.

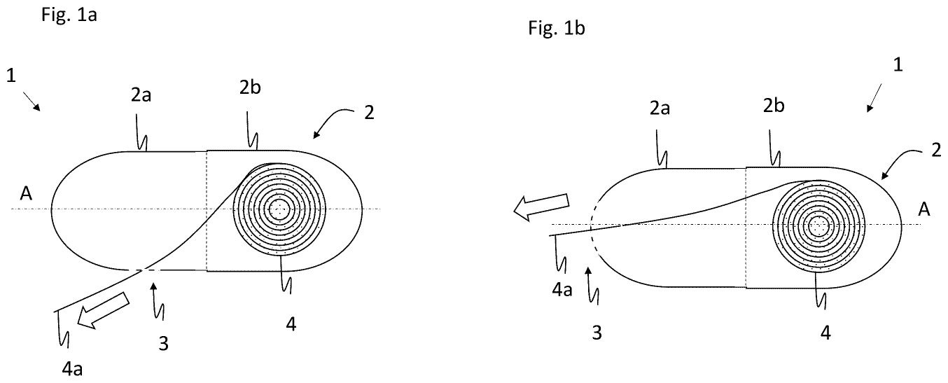

FIGS. 1a, 1b each show schematic illustrations of a capsule device of the drug delivery system.

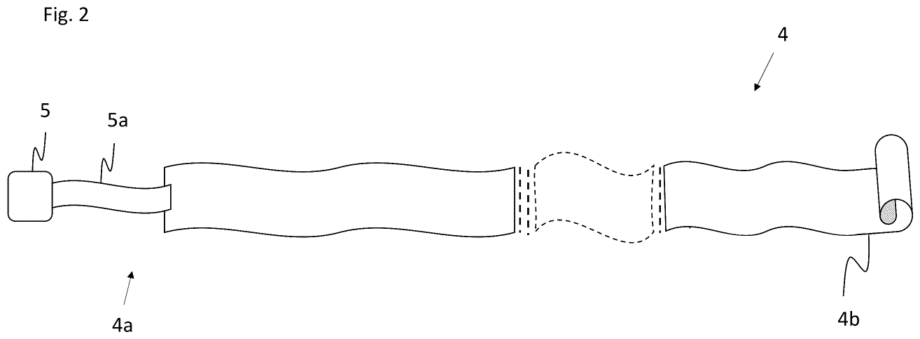

FIG. 2 shows a schematic illustration of a preparation in its partially unfolded form.

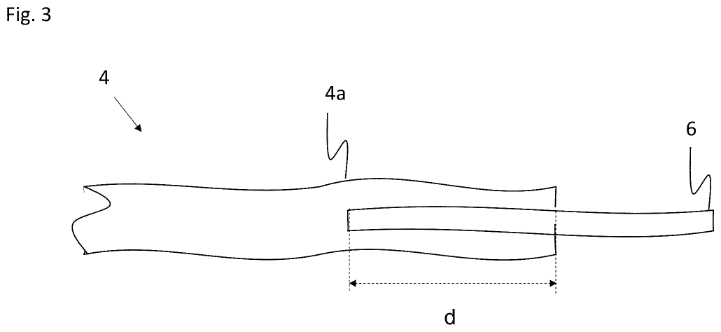

FIG. 3 shows a schematic illustration of the one end of a preparation which is connected to a retainer for pulling the preparation out of the capsule device.

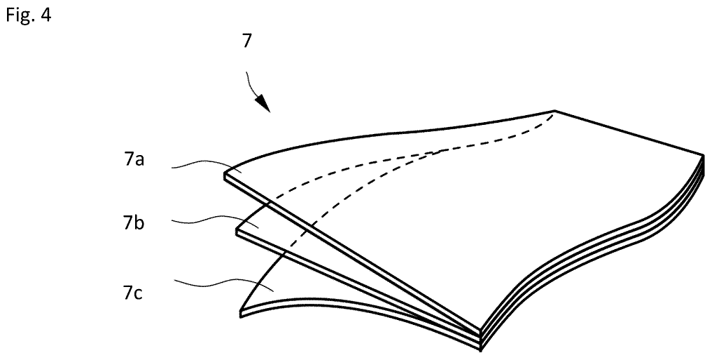

FIG. 4 shows a schematic illustration of the preparation being a three-layered wafer.

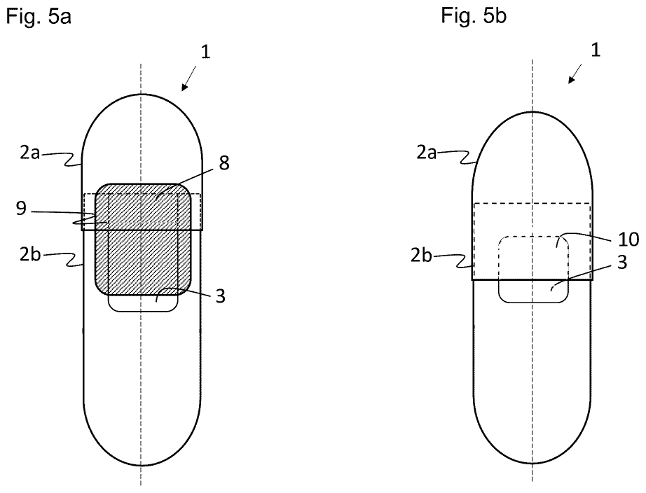

FIGS. 5a, 5b each show schematic illustrations of a capsule device having an aperture formed by an overlapping wall part or by telescoping two capsule halve-shells into each other.

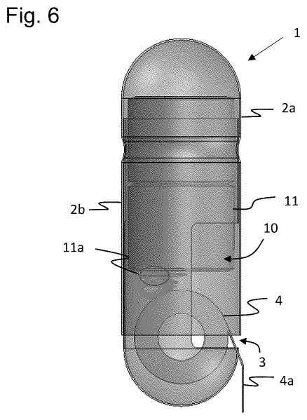

FIG. 6 shows a schematic semi-transparent view of a drug delivery system.

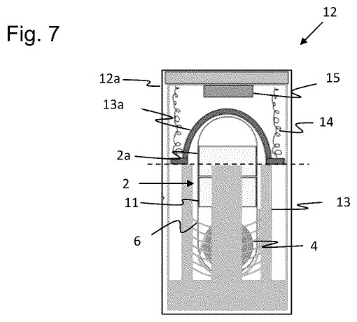

FIG. 7 shows a schematic illustration of an applicator, with a holder and a retainer wound around the holder, whereas a drug delivery system is positioned inside the holder.

FIGS. 8a, 8b each show a schematic view of a patient taking the drug delivery system using the applicator and drinking cup, before (FIG. 8a) and during (FIG. 8b) swallowing of the drug delivery system.

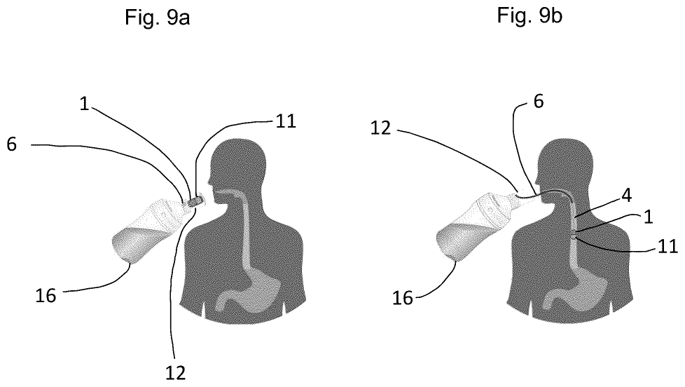

FIG. 9 show a schematic view of a patient taking the drug delivery system including a sinker using the applicator and drinking cup, before (FIG. 9a) and after (FIG. 9b) swallowing of the drug delivery system.

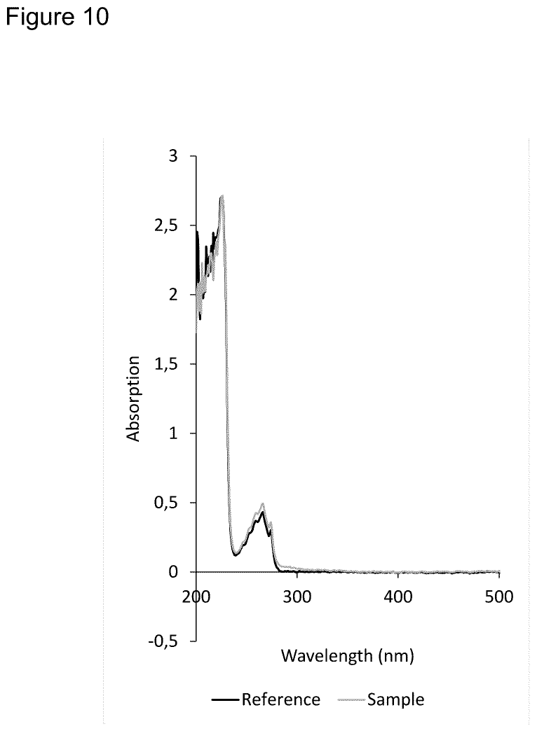

FIG. 10 shows the absorption spectra of Baclofen in a preparation of the drug delivery system (sample) of the invention in comparison to a reference preparation.

FIG. 1a shows a schematic illustration of the drug delivery system 1 having a capsule device 2 with a first halve-capsule shell 2a and a second halve-capsule shell 2b being telescoped into each other thereby forming an aperture 3. The preparation 4 is shown in its compact form inside the capsule 2 with its one end 4a extending out of the aperture 3. An arrow indicated the direction of movement of the preparation 4 when the preparation 4 is pulled out of the capsule 2, i.e., the first halve-capsule shell 2a, through the aperture 3. In FIG. 1a, the aperture 3 is shown formed sidewise to a central axis A of the capsule 2 and arranged in the first halve-capsule shell 2a. In FIG. 2b, the aperture 3 is shown formed along the central axis A of the capsule 2 and arranged in the first-halve capsule shell 2a.

FIG. 2 shows a schematic illustration of a preparation 4 in its partially unfolded form. The preparation 4 is drawn having a sheet like shape. The central area of the preparation 4 is indicated in dashed lines, so that FIG. 2 essentially shows the end 4a of the preparation 4 protruding from the aperture 3 of the capsule device 2 and the still slightly coiled end 4b of the preparation 4. The coiled end 4b indicates the compact form of the preparation 4. At the end 4a, which extends through the aperture 3, a holding device 5 is shown having a patch like shape. The holding device 5 comprises a strip 5a. The strip 5a serves in the embodiment shown in FIG. 2 as connector to link the holding device 5 to the end 4a of the preparation 4. Alternatively, the end 4a of the preparation 4 is directly connected to a retainer, e.g., a string. In such an embodiment, the holding device 5, 5a is formed by the retainer itself.

FIG. 3 shows this preferred embodiment. The end 4a of the preparation 4 is shown having a sheet like shape, whereas a retainer 6 overlaps an end potion having a length d of the preparation 4 to form the holding device 5, 5a. The connection between the retainer 6 and the end 4a of the preparation 4 is made such that a pulling force can be transferred via the connection, when the retainer 6 is tensed to pull the preparation 4 out of the capsule device 2, e.g., by swallowing of the dosage form 1.

FIG. 4 shows the preparation 4 being spilt into several layers. In the shown embodiment of FIG. 4, the preparation is a wafer comprising three distinct layers 7. The one top layer 7a is formed as an adhesive layer, the central layer 7b contains the active pharmaceutical ingredient, and the lower most drawn layer in FIG. 4 shows a protective, e.g., a water protective layer.

FIGS. 5a, 5b each show schematic illustrations of a capsule device 2 of the drug delivery system 1, with an aperture 3 formed by an overlapping wall part 9 or by telescoping two capsule halve-shells 2a, 2b into each other. As shown in FIG. 5a, the first halve-capsule shell 2a is slit over the second halve-capsule shell as indicated by the dashed lines. The second halve-capsule shell comprises a recess 8. By sliding the two halves 2a, 2b partially over each other, the first halve-capsule shell 2a covers the recess 8 of the second halve-capsule shell 2b partially. The further provided wall part 9, then covers the remaining open space formed by the recess 8 such that the aperture 3 is formed as an opening through which the preparation 4 can leave the shell 2.

Alternatively, FIG. 5b shows the embodiment, where the two halve-capsule shells 2a, 2b overlap in a joined position to such an extent that the aperture 3 is formed by the one, in particular, cylindrical wall of the first halve-capsule shell which overlaps the opening 10 of the second halve-capsule shell 2b.

FIG. 6 shows a schematic semi-transparent view of a drug delivery system 1. The first and second halve-capsule shells 2a, 2b are joined in a joined position, thereby form the aperture 3 by covering the opening 10 of the second halve-capsule shell 2b in this position. The end portion 4a of the preparation 4 is shown extending through the aperture 3. The pharmaceutical dosage from 1 further comprises a sinker element 11, which is located in the first halve-capsule shell 2a. The sinker extends from the first-halve capsule shell 2a into the second halve 2b, whereas notches 11a protrude from outside the capsule device 2 into the inside space to position the sinker 11 and to prevent the sinker from moving within the capsule. In FIG. 6 the preparation 4 is shown positioned underneath the sinker 11. The notches prevent the sinker 11 from sliding into the preparation 4.

FIG. 7 shows a schematic illustration of an applicator 12, with a holder 13 and a retainer 6 wound around the holder 13, whereas a drug delivery system 1 is positioned inside the holder 13. The applicator and the holder preferably have a cylindrical shape. The capsule device 2 is positioned inside the holder 13 with the first halve 2a pointing towards applicator cap 12a. The cap 12a is removed for use. In the embodiment shown in FIG. 7, the capsule device 2 further comprises a sinker 11 located in the first halve-capsule shell 2a and the preparation 4 located in the second halve-capsule shell 2b. In the embodiment shown in FIG. 7, the first halve-capsule shell 2a is additionally pressed towards the bottom of the applicator 12. Therefore, a curved holder 13a is positioned above the capsule device 2. The holder 13a is curved such that its shape fits the shape of the first halve-capsule shell 2a. The pressing of the capsule 2 into the holder 13 is achieved by compression springs 14, whose one end is attached to the cap 12a of the applicator 12 and whose other end to the curved holder 13a. A drying element 15 is position inside the applicator 12 at the cap 12a of the applicator 12. This prevents the preparation 4 from being damaged by moisture. The applicator 12 does not necessarily comprise a curved holder 13, a drying element 15 or compression springs 14.

FIGS. 8a, 8b each show a schematic view of a patient taking the drug delivery system 1 using the applicator and drinking cup, before (FIG. 8a) and during (FIG. 8b) swallowing of the drug delivery system. FIG. 8a shows the administration of the drug delivery system comprising the capsule device 2 as herein described by a patient. A drinking cup 16 is filled with a liquid and an applicator 12 is attached to the cup 16. The applicator 12 comprises a retainer 6 and the drug delivery system 1, which further comprises the capsule device 2, connected to the preparation 4, and which is at least partially coiled at the inside of the capsule device 2. FIG. 8b illustrates the procedure when the patient swallows the dosage form 1 and the dosage form 1 then is transported through the esophagus towards the stomach. The retainer 12 pulls the preparation 4 out of the capsular device 2. The preparation 4 then spreads along the esophagus so that the active ingredient of the dosage form 1 is delivered to the mucosa of the esophagus.

### LIST OF REFERENCE SYMBOLS

1 drug delivery system
2 capsule device
2a first halve-capsule shell
2b second halve-capsule shell
3 aperture
4 preparation
4a end of the preparation that extends through the aperture
4b coiled end of the preparation
5 holding device
5a strip
6 retainer
7 layer
7a adhesive layer
7b active pharmaceutical ingredient-containing layer
7c protective layer
8 recess
9 wall part
10 opening
11 sinker element
11a notches
12 applicator
12a applicator cap
13 holder
13a curved holder
14 spring
15 drying element
16 drinking cap

### EXAMPLES

### Example 1: Preparation of a Polymer Film with 4-Amino-3-(4-chlorphenyl) Butyric Acid (Baclofen)

A “base polymer mixture” was prepared with the ingredients and the amount as depicted in Table 1 without addition of Baclofen.

TABLE 1

| Ingredient | Amount (g) | Proportion Total | Proportion Solid |
|---|---|---|---|
| PVA 18-88 | 18.0 | 17% | 80% |
| Glycerol | 2.0 | 2% | 9% |
| Dem. Water | 80.0 | 76% | — |
| 0.1M HCl | 2.5 | 2% | — |
| Baclofen | 2.5 | 2% | 11% |

After pH adjustment, Baclofen was added at room temperature and film laminates were prepared by solvent casting, i.e., the polymer mixture comprising Baclofen was spread and the solvent was evaporated off. The films were dried at room temperature.

The resulting films were flexible and showed an optically homogenous, air bubble free surface. Further, the surface of the film was uniformly rough with particle sized of different dimensions.

Further, the film was analyzed for the content of Baclofen. To this end, 6 circular samples with a diameter of 1.9 cm (2.84 cm²) were cut at random positions of the film and analyzed with a UV/Vis spectroscopy in FSG medium, pH 1.2. The absorption spectrum is shown in FIG. 10 in comparison to a reference sample comprising Baclofen solved in FSG medium, pH 1.2. Comparison of the spectrum of the film sample with the spectrum of the reference sample confirms that the Baclofen can be stably prepared in a film for use in the drug delivery device of the invention.

Further, the content of Baclofen on the film was as indicated in Table 2.

TABLE 2

| | Baclofen per film | Baclofen per area | Film mass per |
|---|---|---|---|
| | mass (mg/mg) | (mg/cm²) | area (g/m²) |
| Mean value | 9% | 1.40 | 148 |
| SD* | 0% | 0.07 | 8 |
| rel. SD** | 1% | 5% | 5 |

*Standard Deviation,
**relative Standard Deviation

The results in Table 2 show that loading of 10 cm² of the film with 15 mg Baclofen is possible and allowing the preparation of a film with a wide therapeutic dosage range. It must be noted that standard deviation and maximum of drug load are determined by the process technology and do not represent any limitations in principle.

Moreover, oral administration of Baclofen in the art, i.e., by absorption by the digestive system to enter the blood stream, requires high doses of 15 to 75 mg per day. Further, oral administration of Baclofen, in clinical use is very limited because of its poor tolerability due to severe side effects, such as neurological adverse events. Common side effects include sleepiness, weakness, headache, confusion, nausea, and dizziness. Topical and/or local administration of Baclofen via the drug delivery system of the invention allows for improved local administration to the target site thereby increasing the value of this drug, particularly in the treatment of esophageal diseases.

### Example 2: Preparation of a Polymer Film by Melt Extrusion

Selecting the appropriate polymer base: choose a polymer base that is compatible with the active ingredients you intend to use. Typical polymers are polyvinyl alcohol (PVA), cellulose ether and polyethylene glycol (PEG) in combination with suitable plasticizers.
Active ingredient preparation: The active ingredient is usually prepared in a suitable formulation (e.g., as powder or granules).
Mixing of the components: The polymer base and the active ingredient are mixed in a mixer to form a homogeneous mixture.
Extrusion: The mixture is fed into an extruder, where it is melted at high temperatures and forced through a die. The die forms the film, which is deposited on a cooling plate.
After the polymer compound is forced through the die during melt extrusion, the extruded film can undergo a rolling process to further optimize its thickness and properties. The rolling process can include either cold or hot rolling, depending on the specific requirements of the film.
Post-treatment: After the extrusion process, the extruded film is cut to the desired size and shape. The film is then usually subjected to further processes such as drying, coating or laminating to improve its physical and pharmaceutical properties.

1. A drug delivery system for the application to an esophageal mucous membrane, comprising
at least one sheet like preparation comprising an active pharmaceutical ingredient
a release mechanism; and
a trigger mechanism, wherein
the trigger mechanism is adapted to trigger, at a predetermined site of action, the release of the preparation by the release mechanism, and wherein the release mechanism is adapted to release said preparation while moving along the esophageal mucous membrane,
wherein the drug delivery system further comprises a shell, wherein the shell contains the preparation, and wherein the shell comprises an aperture as part of the release mechanism configured to allow said preparation to leave the shell, and wherein the trigger mechanism is a holding device that is a part of or is attached to the preparation, such that the preparation is unrolled or unfolded while the dosage form moves down the esophageal mucous membrane and leaves the shell through the aperture,
characterized in that the active pharmaceutical ingredient comprises a reflux inhibitor.

2. The drug delivery system of claim 1, wherein the reflux inhibitor comprises a GABA (gamma-Aminobutyric acid) receptor type B agonist.

3. The drug delivery system of claim 2, wherein the GABA receptor type B agonist is selected from the group consisting of gamma-Aminobutyric acid (GABA) or a salt thereof, β-Phenyl-γ-aminobutyric acid (Phenibut) or a salt thereof, 4-Fluorophenibut or a salt thereof, Isovaline or a salt thereof, 3-Aminopropylphosphinic acid or a salt thereof, 3-Aminopropylmethylphosphinic acid (SKF-97,541) or a salt thereof, [3-Amino-2-hydroxypropyl]-methylphosphinic acid (CGP 44532) or a salt thereof, 4-Amino-3-(4-chlorphenyl) butyric acid (Baclofen) or a salt thereof, [(2R)-3-Amino-2-fluoropropyl] phosphinic acid (Lesogaberan) or a salt thereof, [2,6-di-tert-butyl-4-(3-hydroxy-2,2-dimethyl-propyl)-phenol] (CGP7930) or a salt thereof, (S)-3-amino-2-hydroxypropylphosphinic acid (AZD9343) or a salt thereof, and Arbaclofen placarbil or a salt thereof.

4. The drug delivery system of claim 2, wherein the GABA receptor type B agonist comprises 4-Amino-3-(4-chlorphenyl) butyric acid (Baclofen) or a salt thereof.

5. The drug delivery system of claim 2, wherein the GABA receptor type B agonist comprises the R-enantiomer of 4-Amino-3-(4-chlorphenyl) butyric acid (Baclofen) or a salt thereof.

6. The drug delivery system of claim 1, wherein the sheet like preparation comprising the active pharmaceutical ingredient comprises polyvinyl alcohol (PVA).

7. A method of treating or preventing an esophageal disease in a patient, comprising administering the drug delivery system of claim 1 to a patient.

8. The method of claim 7, wherein the esophageal disease is caused by or related to gastroesophageal reflux.

9. The method of claim 8, wherein the esophageal disease is caused by or related to gastroesophageal reflux disease (GERD), non-erosive reflux disease (NERD), esophagitis, particularly erosive esophagitis (EE) and/or cancer.

10. A method of treating or preventing gastroesophageal reflux disease in a patient, comprising administering the drug delivery system of claim 1 to the patient.

11. The drug delivery system of claim 1, wherein:
a) the sheet like preparation is a film shaped, foil shaped or wafer shaped preparation; or
b) the reflux inhibitor reduces or blocks transient lower esophageal reflux relaxations (TLESRs).

12. The method of claim 8, wherein the esophageal disease is Barrett's esophagus (BE).

13. The method of claim 9, wherein the esophageal disease is cancer.

14. The method of claim 13, wherein the esophageal disease is adenocarcinoma.
